(19) United States
(12) Patent Application Publication
(10) Pub. No.: US 2012/0253321 A1
(43) Pub. Date: Oct. 4, 2012

(54) GUIDEWIRE

(75) Inventor: Atsushi TSUNEZUMI, Nagoya-shi (JP)

(73) Assignee: ASAHI INTECC CO., LTD., Nagoya-shi (JP)

(21) Appl. No.: 13/427,117

(22) Filed: Mar. 22, 2012

(30) Foreign Application Priority Data
Mar. 30, 2011 (JP) 2011-074103

(51) Int. Cl. A61M 25/09

(52) U.S. Cl. 604/528

(57) ABSTRACT

A guidewire includes a core shaft and a coil body that covers at least a distal end portion of the core shaft. Along a center line in a longitudinal-axis direction of the core shaft, the distal end portion of the core shaft has a first pre-assembly curvature with respect to a proximal end of the core shaft. Along a center line in a longitudinal-axis direction of the coil body, at least a distal end portion of the coil body has a second pre-assembly curvature with respect to a proximal end of the coil body. The first pre-assembly curvature is less than the second pre-assembly curvature. When the core shaft and the coil body are coupled to each other, a distal end portion of the guidewire has, along a center line in a longitudinal-axis direction of the guidewire, a third curvature with respect to a proximal end of the guidewire.

### CROSS-REFERENCE TO RELATED APPLICATION

This application claims priority to Japanese Patent Application No. 2011-074103 filed in the Japan Patent Office on Mar. 30, 2011, the entire contents of which are incorporated herein by reference.

### BACKGROUND

The disclosed embodiments relate to a medical device. More specifically, the disclosed embodiments relate to a guidewire. A guidewire is used as a guide when inserting a catheter into, for example, a blood vessel, a ureter, or an organ, or when inserting an indwelling device into an aneurysm of a blood vessel. Ordinarily, the guidewire includes a core shaft (core wire) and a coil body (coil) that is wound around an outer surface of a distal end portion of the core shaft. A distal end portion of the coil body and the distal end portion of the core shaft are joined to each other to form a tip portion.

Further, in order to increase maneuverability when reaching a side branch of a blood vessel, a distal end of the guidewire may be curved. For example, Japanese Unexamined Patent Application Publication No. 7-255856 (Patent Document 1) discloses a method in which a distal end portion of a coil and a distal end portion a core wire of the guidewire are inserted into a die having a J-shaped recessed portion to cause the distal end portion of the coil and the distal end portion of the core wire to undergo plastic deformation.

### SUMMARY

Since the distal end portion of the guidewire that is described in Patent Document 1 has a curved shape whose curvature is substantially the same as that of the distal end portion of the core wire and that of the distal end portion of the coil, the distal end portion of the guidewire has excellent shape-keeping properties. However, when the distal end portion of the guidewire during use is elastically deformed into a linear shape, what is called “jumping” of the distal end portion occurs excessively due to resilient restoring force resulting from the elastic deformation.

Accordingly, in view of such problems, it is an object of the present invention to make it possible to provide improved maneuverability by preventing excessive jumping while providing a shape-keeping property of a distal end portion of a guide wire.

According to an embodiment of the present invention, there is provided a guidewire including a core shaft having a distal end portion and a proximal end, and a coil body that covers at least the distal end portion of the core shaft and that has a distal end portion and a proximal end. In the guidewire, along a center line in a longitudinal-axis direction of the core shaft, the distal end portion of the core shaft has a first curvature with respect to the proximal end of the core shaft. Along a center line in a longitudinal-axis direction of the coil body, at least the distal end portion of the coil body has a second curvature with respect to the proximal end of the coil body. The first curvature is less than the second curvature. When the core shaft and the coil body are coupled to each other, a distal end portion of the guidewire has, along a center line in a longitudinal-axis direction of the guidewire, a third curvature with respect to a proximal end of the guidewire. The third curvature is less than the second curvature.

When the distal end portion of the guidewire having the above-described structure has a shape having a third curvature after the core shaft and the coil body have been coupled to each other, the third curvature of the shape of the distal end portion of the core shaft is greater than the first curvature of the shape of the distal end portion of the core shaft prior to coupling thereof to the coil body (that is, the distal end portion of the core shaft is deformed into a shape having a tighter curve than it had prior to assembly), whereas the third curvature of the shape of the distal end portion of the coil body becomes less than the second curvature of the shape of the distal end portion of the coil body prior to coupling thereof to the core shaft (that is, the distal end portion of the coil body is deformed into a more linear shape than it had prior to assembly). Therefore, when the guidewire is inserted into, for example, a stenotic lesion of a blood vessel, if the distal end portion of the curved shape is deformed into a linear shape, the core shaft itself approaches its original shape. Therefore, the amount of deformation of the core shaft is reduced, as a result of which resilient restoring force generated at the core shaft is reduced. Consequently, when the distal end portion of the guidewire is deformed into a linear shape, jumping of the core shaft is reduced, so that excessive jumping of the guidewire as a whole is prevented from occurring.

The disclosed embodiments make it possible to provide shape-keeping property of the distal end portion of the guidewire, and to prevent excessive jumping to thereby provide excellent maneuverability of the guidewire.

### BRIEF DESCRIPTION OF THE DRAWINGS

FIG. 1 illustrates a guidewire.

FIG. 2A illustrates a core shaft.

FIG. 2B illustrates a coil body.

FIG. 3 illustrates a guidewire according to another embodiment.

FIG. 4 illustrates a guidewire according to still another embodiment.

FIGS. 5A and 5B illustrate a core shaft having another form.

FIG. 6 illustrates a core shaft having another form.

FIG. 7 comparatively illustrates the pre-assembly radii of curvature of the core shaft and the coil body and the radius of curvature of the assembled guide wire.

### DETAILED DESCRIPTION OF EMBODIMENTS

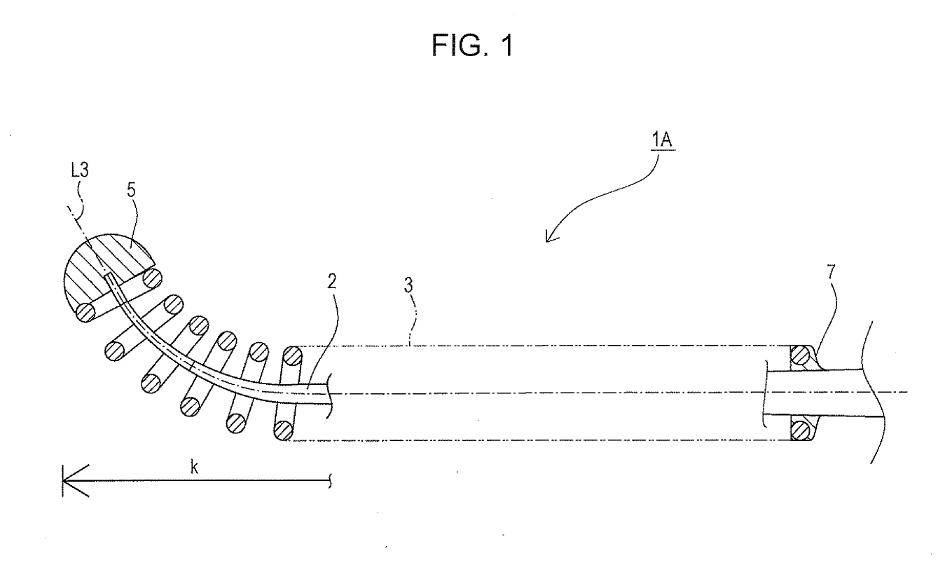

As shown in FIG. 1, a guidewire 1A includes a core shaft 2 and a coil body 3, which are coupled to each other. To increase maneuverability of the guidewire, a distal end portion k has a curved shape.

First, an external shape of the core shaft 2 and an external shape of the coil body 3 will be described in detail.

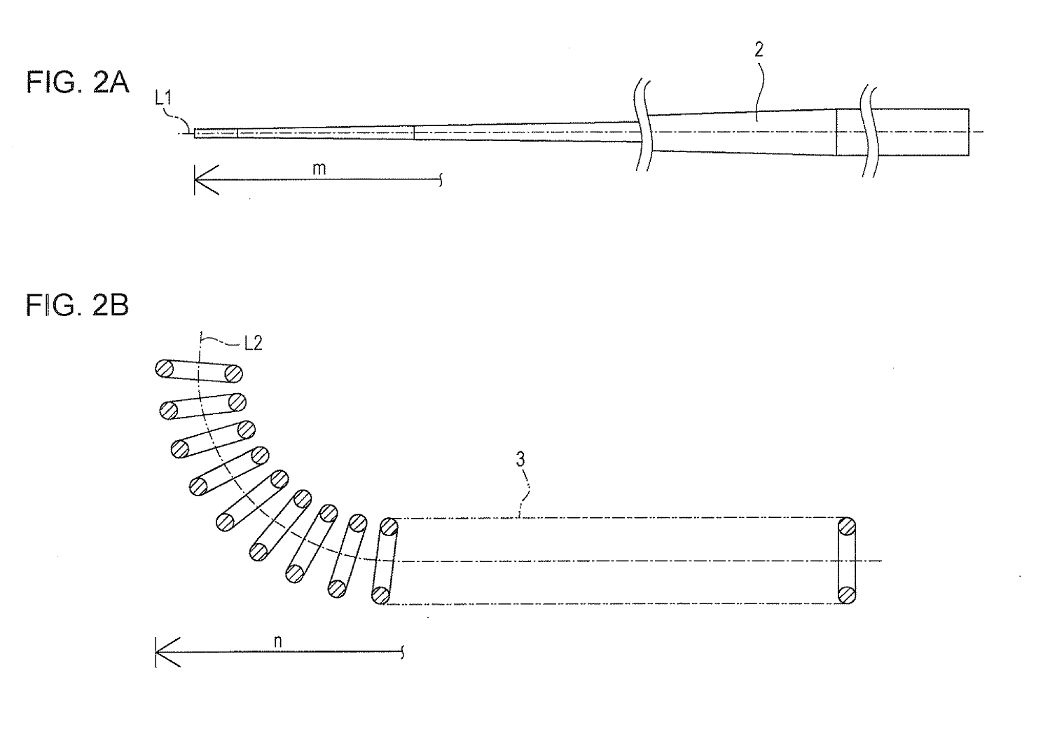

As shown in FIG. 2A, prior to coupling, the core shaft 2 has the shape of a tapered round bar whose distal-end side has a small diameter and whose proximal-end side has a larger diameter, and has what is called a linear shape over its entire length. That is, along a center line L1 in a longitudinal-axis direction of the core shaft 2, a curvature X1 at least at a distal end portion m is equal to zero (X1=0) with respect to a proximal end of the core shaft 2. That is, the core shaft 2 has a substantially linear (straight line) shape prior to coupling the core shaft 2 to the coil body 3.

As shown in FIG. 2B, the coil body 3 is sparsely wound, and at least a distal end portion n of the coil body 3 is previously formed into a curved shape prior to coupling of the coil body 3 to the core shaft 2. That is, along a center line L2 in a longitudinal-axis direction of the coil body 3, a curvature X2 at least at the distal end portion n is set in a range of at least X2>0 with respect to a proximal end of the coil body 3. Here, when the curvature X2 is compared with the curvature X1 at the distal end portion m of the core shaft 2, the curvature X1 is less than the curvature X2.

The core shaft 2 and the coil body 3 are each formed of, for example, a stainless alloy such as SUS304 or SUS316.

As shown in FIG. 1, the coil body 3 covers the distal end portion m of the core shaft 2, the distal end portion m of the core shaft 2 and the distal end portion n of the coil body 3 are secured to each other by a tip portion 5 having a substantially semispherical shape, and the proximal end of the coil body 3 is secured to the core shaft 2 at a securing portion 7, so that the guidewire 1A is formed. The tip portion 5 and the securing portion 7 are formed for example, by bonding using an adhesive, soldering, or welding materials.

Here, as shown in FIG. 1, the guidewire 1A in which the core shaft 2 and the coil body 3 are coupled to each other is such that, along a center line L3 in the longitudinal-axis direction of the guidewire 1A, a curvature X3 at the distal end portion k is at least in a range of X3>0 with respect to the proximal end of the guidewire 1A. More specifically, the curvature X1 at the distal end portion m of the core shaft 2, the curvature X2 at the distal end portion n of the coil body 3, and the curvature X3 at the distal end portion k of the guidewire 1A satisfy the following relationship:

X1(=0)<X3<X2

Here, the curvature X1 of the core shaft 2 corresponds to a first curvature according to an embodiment of the present invention. The curvature X2 of the coil body corresponds to a second curvature. The curvature X3 of the guidewire 1A corresponds to a third curvature.

Since the distal end portion k of the guidewire 1A is curved, the guidewire 1A provides excellent maneuverability in, for example, a blood vessel. In the case where the guidewire 1A is inserted into, for example, a stenotic lesion, when the distal end portion k is deformed into a linear shape, the core shaft 2 itself approaches its original shape. Therefore, the amount of deformation of the core shaft 2 is reduced, as a result of which resilient restoring force generated at the core shaft 2 is reduced. Consequently, even if the distal end portion k of the guidewire 1A is deformed into a linear shape, jumping of the core shaft 2 is reduced, so that excessive jumping of the guidewire 1A as a whole is prevented from occurring. In addition, since the core shaft 2 and the coil body 3 are each formed of a stainless alloy, a doctor can finely adjust the amount of curvature of the distal end portion k of the guidewire 1A, to adjust the distal end portion k to a desired curved shape. Further, since the stainless alloy is highly rigid, rotation transmission ability of the distal end portion k of the guidewire 1A is increased, so that maneuverability of the guidewire 1A is further increased.

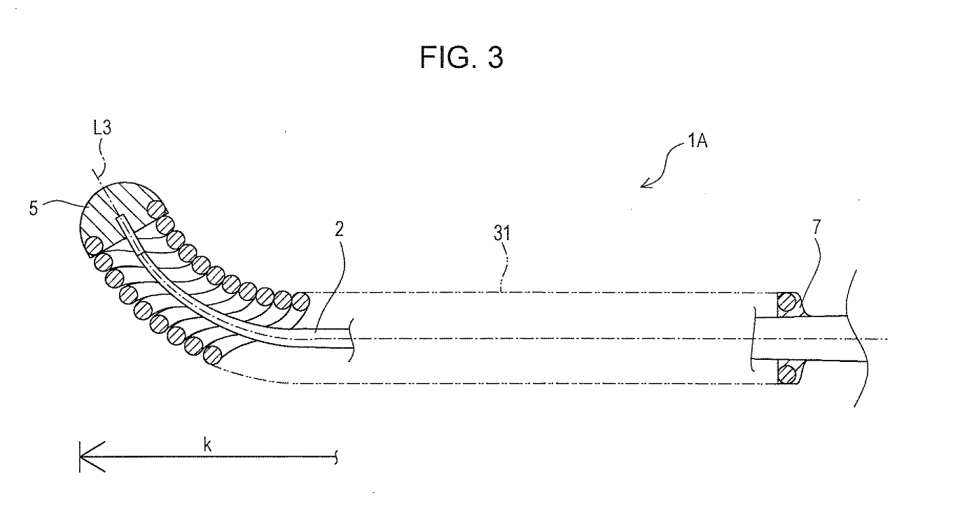

The coil body 3 is not limited to a single coil. As shown in FIG. 3, a coil body 31 including a multi-thread coil may be used. The multi-thread coil has excellent resiliency. Therefore, the curved shape of the distal end portion k of the guidewire 1A when the guidewire 1A is used is reliably restored, so that the maneuverability is reliably maintained. In addition, since the multi-thread coil is such that a plurality of coil wires are twisted and wound, even if the curved shape is previously formed, gaps are not easily formed between the coil wires. Therefore, when the coil body 31 is coupled to the core shaft 2, the core shaft 2 is easily inserted into the coil body 31, so that the mounting can be smoothly performed. Consequently, the manufacturing process of the guidewire 1A is streamlined.

Some ways in which the above-described structures may be modified are described below. Portions that correspond to those of the above-described embodiments will not be described below. They will be given the same reference numerals in the figures.

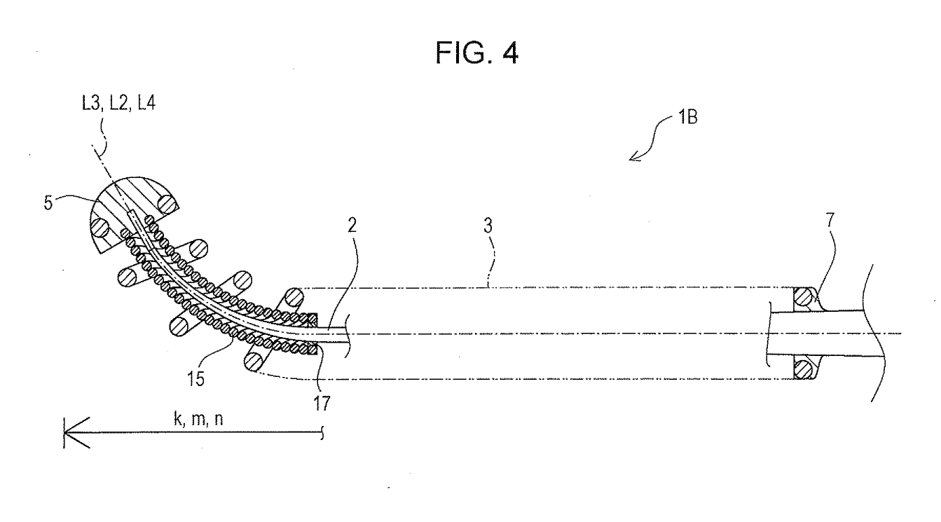

As shown in FIG. 4, a guidewire 1B according to another embodiment includes a linear core shaft 2 and a coil body 3 including a single coil. An inner coil body 15 that covers at least a distal end portion m of the core shaft 2 is provided at an inner side of a distal end portion n of the coil body 3. The inner coil body 15 includes a multi-thread coil. A distal end of the inner coil body 15 is caused to adhere to the aforementioned tip portion 5, and a proximal end of the inner coil body 15 is secured to the core shaft 2 at a securing portion 17. The securing portion 17 is suitably formed in the same manner described above as that used to form the tip portion 5 and the securing portion 7. The inner coil body 15 is previously formed into a curved shape. A curvature X4 along a center line L4 in a longitudinal-axis direction of the inner coil body 15 prior to mounting thereof is set so as to be equal to a curvature X2 along a center line L2 at a distal end portion n of the coil body 3 at the outer side.

When the guidewire 1B includes the inner coil body 15, plastic deformation of a distal end portion k of the guidewire 1B is stably suppressed due to the restoring force of the inner coil body 15. Therefore, the maneuverability of the guidewire 1B is even further increased. The multi-thread coil of the inner coil body 15 has excellent resiliency. Therefore, the curved shape of the distal end portion k of the guidewire 1B when the guidewire 1B is used is reliably restored, so that the maneuverability is reliably maintained. In addition, when the multi-thread coil is formed into a curved shape, gaps are not easily formed between the coil wires. Therefore, when the inner coil body 15 is coupled to the core shaft 2, the core shaft 2 is easily inserted into the inner coil body 15, so that the mounting can be smoothly performed. Consequently, the manufacturing process of guidewires 1B is streamlined.

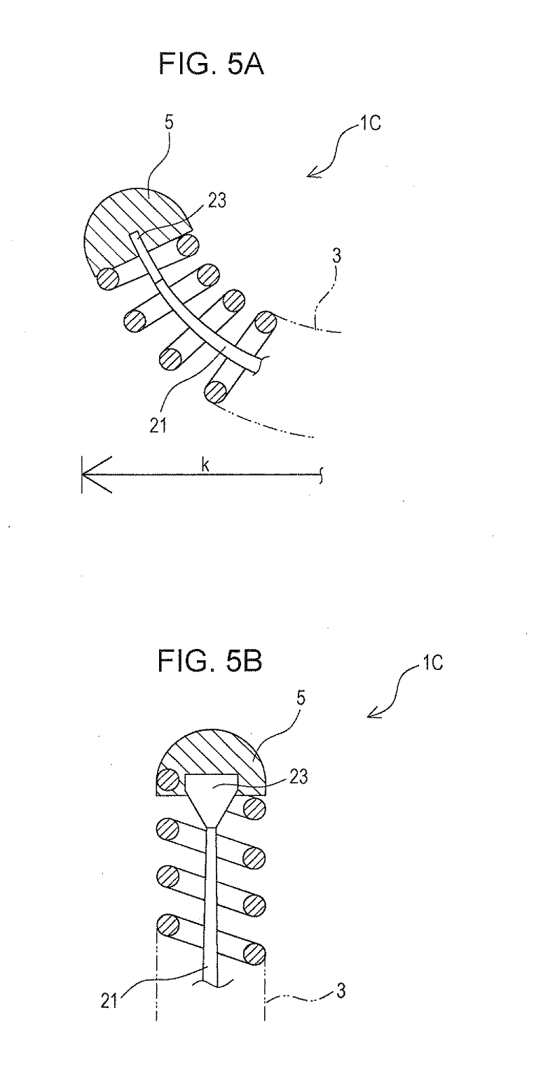

As shown in FIGS. 5A and 5B, a distal end portion 23 of a core shaft 21 may be formed into a flattened shape. The core shaft 21 in the coupled state is disposed so that one of the flat surfaces of the distal end portion 23 faces the center of curvature of a guidewire 1C. Such a structure makes it possible to orient the distal end portion of the guidewire 1C, so that shaping of the guidewire 1C is more easily performed. Therefore, a doctor can finely adjust the amount of curvature of the distal end portion k (see FIG. 5A) of the guidewire 1C, to adjust a distal end portion k to a desired curved shape. As a result, the maneuverability of the guidewire 1C is further increased.

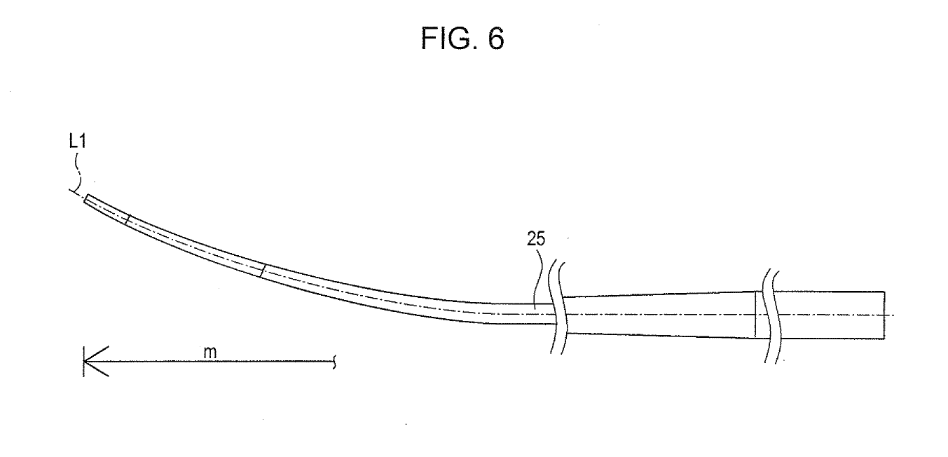

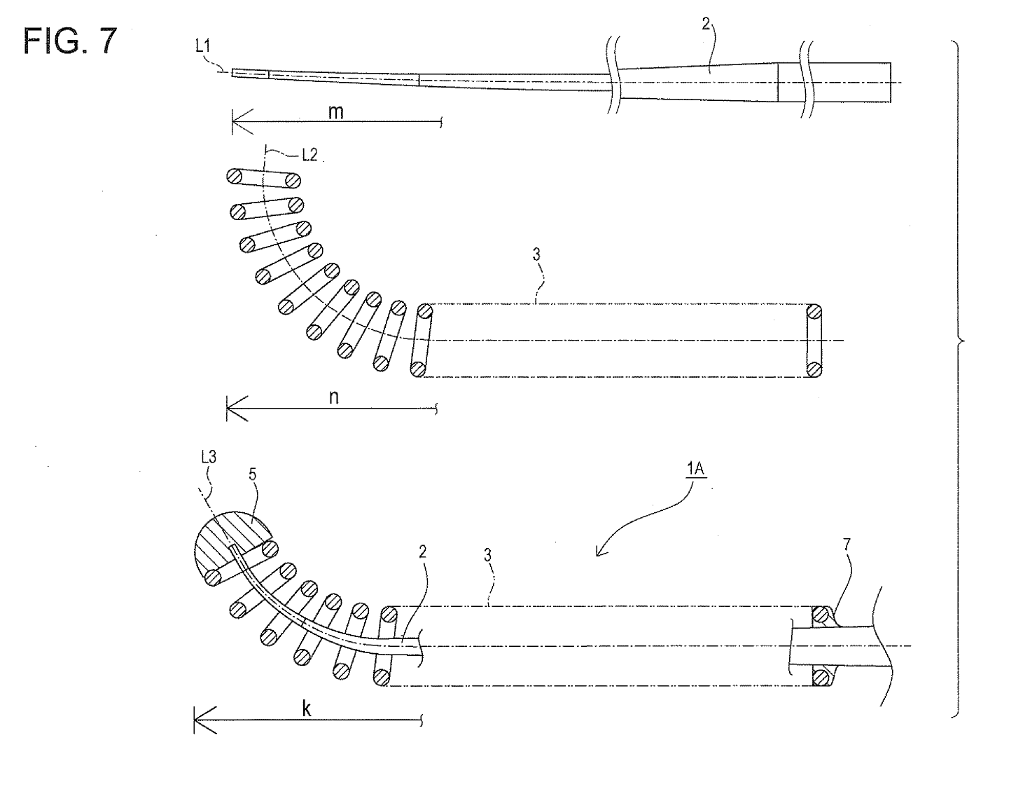

As shown in FIG. 6, a distal end portion m of a core shaft 25 prior to mounting thereof may be previously formed into a curved shape. More specifically, a curvature X1 at the distal end portion m of the core shaft 25 satisfies the following relationship in an embodiment of the present invention:

0<X1<X3<X2

The core shafts 2, 21, and 25, and the coil bodies 3 and 31 described thus far may be formed of pseudoelastic alloys such as NiTi. This enhances shape-restoring properties of the core shafts 2, 21, and 25, and the coil bodies 3 and 31. Therefore, plastic deformation of the distal end portion k of each of the guidewires 1A to 1C is suppressed, thereby further increasing maneuverability.

While the foregoing embodiments have been shown and described in detail, the foregoing description is in all aspects illustrative and not restrictive. It is therefore understood that numerous modifications and variations can be devised without departing from the spirit and scope of the present invention.

For example, the curved shape of the distal end portion k of each of the guidewires 1A to 1C may be modified as appropriate. The curved shape may be, for example, an angular shape or a J shape. It is possible for the core shaft and the coil bodies 3 and 31 to have what is called a linear shape over the entire length thereof, and for only the inner coil body 15 to have a curvature. When only the inner coil body 15 has a curvature, the inner coil body 15 is desirably a multi-thread coil body. In addition, the coil bodies 3 and 31 are not limited to those in which only the distal end portion n is formed into a curved shape. The whole coil body 3 and the whole coil body 31 may be formed into a curved shape (such as a C shape). Essentially, the distal end portion of the coil body 3 and the distal end portion of the coil body 31 may have any curved shape as long as the curved shape has the predetermined curvature X2 with respect to the center line L2 at the proximal end of the coil body 3 and the proximal end of the coil body 31.

What is claimed is:

1. A guidewire comprising:
a core shaft having a distal end portion and a proximal end portion; and
a coil body having a distal end portion and a proximal end portion, the coil body covering at least the distal end portion of the core shaft, wherein
along a center line in a longitudinal-axis direction of the core shaft, the distal end portion of the core shaft has a first pre-assembly curvature with respect to the proximal end portion of the core shaft,
along a center line in a longitudinal-axis direction of the coil body, at least the distal end portion of the coil body has a second pre-assembly curvature with respect to the proximal end portion of the coil body,
the first pre-assembly curvature is less than the second pre-assembly curvature, and
after the core shaft and the coil body have been coupled to each other, a distal end portion of the guidewire has, along a center line in a longitudinal-axis direction of the guidewire, a third curvature with respect to a proximal end of the guidewire, the third curvature being less than the second pre-assembly curvature.

2. The guidewire according to claim 1, wherein the coil body is a multi-thread coil including a plurality of wires.

3. The guidewire according to claim 1, wherein an inner side of at least the distal end portion of the coil body is provided with an inner coil body that covers at least the distal end portion of the core shaft, the inner coil body having a distal end portion and a proximal end portion, and
wherein, along a center line in a longitudinal-axis direction of the inner coil body, at least the distal end portion of the inner coil body has the second pre-assembly curvature with respect to the proximal end of the inner coil body.

4. The guidewire according to claim 3, wherein the inner coil body is a multi-thread coil including a plurality of coil wires.

5. The guidewire according to claim 1, wherein the core shaft and the coil body are each formed of a stainless alloy.

6. The guidewire according to claim 3, wherein the inner coil body is formed of a stainless alloy.

7. The guidewire according to claim 1, wherein the core shaft and the coil body are each formed of a pseudoelastic alloy.

8. The guidewire according to claim 1, wherein the first pre-assembly curvature of the core shaft is zero.

9. The guidewire according to claim 3, wherein the first pre-assembly curvature of the core shaft is zero.

10. The guidewire according to claim 1, wherein the third curvature of the guidewire is greater than the first pre-assembly curvature of the core shaft.

11. The guidewire according to claim 3, wherein the third curvature of the guidewire is greater than the first pre-assembly curvature of the core shaft.
